(19) United States
(12) Patent Application Publication
(10) Pub. No.: US 2022/0246305 A1
(43) Pub. Date: Aug. 4, 2022

(54) GENERATION OF DATASETS FOR MACHINE LEARNING MODELS USED TO DETERMINE A GEO-LOCATION BASED LIFESCORE

(71) Applicants: AVANEENDRA GUPTA, CUPERTINO, CA (US); ASHOK BARDHAN, el cerrito, CA (US)

(72) Inventors: AVANEENDRA GUPTA, CUPERTINO, CA (US); ASHOK BARDHAN, el cerrito, CA (US)

(21) Appl. No.: 17/467,139

(22) Filed: Sep. 3, 2021

Related U.S. Application Data
(60) Provisional application No. 63/074,468, filed on Sep. 3, 2020.

(51) Int. Cl. G16H 50/30; G16H 50/70

(52) U.S. Cl. CPC G16H 50/30; G16H 50/70

(57) ABSTRACT

In one aspect, a computerized method for generation of datasets for machine learning models is used to determine a geo-location-based LifeScore. The method includes the step of implementing a machine learning modeling process to combine a healthcare attribute variable as an outcome or target variable as a function of lifestyle behavioral attributes, socio-economic, demographic, healthcare provisional, socio-networking, physical-environmental and other locality specific feature variables to generate a life outcome model of a locality. The method includes the step of updating the life outcome model based on a combination of location specific mortality, life expectancy, self assessed poor health variable value, a poor physical health days variable value, a frequent physical distress variable value. The method includes the step of using a set of socio-economic wellbeing principal components on the independent, driving side of features to update the life outcome model. The method includes the step of generating a community well-being index of the locality to update the life outcome model. The method includes the step of using a set of variables that measure collective efficacy or social cohesion to update the life outcome model. The method includes the step of using a specified community, institutional and family index to update the life outcome mode. The method includes the step of using the life outcome model to generate a LifeScore.

### CLAIM OF PRIORITY

This application claims priority to U.S. Provisional Patent Application No. 63,074,468, filed on Sep. 03, 2020. This provisional application is incorporate herein by reference.

### FIELD OF THE INVENTION

The present invention is in the field of machine learning and more particularly to the generation of datasets for training of machine learning systems.

### BACKGROUND

A person's overall health and how long they live can depend on their physical and social surroundings as much as on their genetic make up and their individual lifestyle—nutrition, exercise—choices. To a considerable extent, a person's locality, neighborhood, town, or city determine their exercise regimen and food choices, their nutritional intake, their social support structure, and their health care access resulting in significant impact on life and health outcomes. This happens both through a demonstration effect as well as supply side, provision effects. On the positive side, this occurs because social interaction and networks, social relationships and community connections can help provide mutual aid and support. This can be particularly vital at a time of a health crisis.

There is increasing recognition that social connections among individuals have a salutary effect on many kinds of outcomes, including health related. Some specific features of localities and neighborhoods around the home—the socio-cultural ethos, economic and community health, social and civic engagement—have a positive impact on health and in battling disease spread—Covid, for example. These external, social determinants of life and health can play a role in determining life expectancy and mortality rate, over and above individual-specific physical, genetic, behavioral, and other vulnerabilities. Accordingly, improvement to the generation of datasets for machine learning models used to determine a geo-location-based LifeScore are desired.

### SUMMARY OF THE INVENTION

In one aspect, a computerized method for generation of datasets for machine learning models is used to determine a geo-location-based LifeScore. The method includes the step of implementing a machine learning modeling process to combine a healthcare attribute variable as an outcome or target variable as a function of lifestyle behavioral attributes, socio-economic, demographic, healthcare provisional, socio-networking, physical-environmental and other locality specific feature variables to generate a life outcome model of a locality. The method includes the step of updating the life outcome model based on a combination of location specific mortality, life expectancy, self assessed poor health variable value, a poor physical health days variable value, a frequent physical distress variable value. The method includes the step of using a set of socio-economic wellbeing principal components on the independent, driving side of features to update the life outcome model. The method includes the step of generating a community well-being index of the locality to update the life outcome model. The method includes the step of using a set of variables that measure collective efficacy or social cohesion to update the life outcome model. The method includes the step of using a specified community, institutional and family index to update the life outcome mode. The method includes the step of using the life outcome model to generate a LifeScore.

### BRIEF DESCRIPTION OF THE DRAWINGS

FIG. 1 illustrates an example process for data set generation and modelling for determining a LifeScore, according to some embodiments.

FIG. 2 illustrates an example process for model implementation according to some embodiments.

FIG. 3 is a block diagram of a sample computing environment that can be utilized to implement various embodiments.

FIG. 4 is a schematic representation of an exemplary hardware environment for generation of datasets for machine learning models used to determine a geo-location-based LifeScore.

FIG. 5 illustrates an example of a webpage snapshot of a an example of the county-wise data frames that can be utilized herein, according to some embodiments.

FIG. 6 illustrates an example process for generation of datasets for machine learning models used to determine a geo-location-based LifeScore, according to some embodiments.

FIG. 7 illustrates an example screen shot of correlations for a LifeScore, according to some embodiments.

FIG. 8 illustrates an example chart illustrating how LifeScores are correlated across age groups and gender, according to some embodiments.

FIG. 9 illustrates screenshot of LifeScore web map, according to some embodiments.

The Figures described above are a representative set and are not an exhaustive with respect to embodying the invention.

### DESCRIPTION

Disclosed are a system, method, and article of manufacture for generation of datasets for machine learning models used to determine a geo-location-based LifeScore. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment;’ ‘an embodiment,’ ‘one example,’ or similar language means that a particular feature, structure, or characteristic described in connection with the embodiment is included in at least one embodiment, according to some embodiments. Thus, appearances of the phrases ‘in one embodiment;’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Definitions

Example definitions for some embodiments are now provided.

Generalized linear model (GLM) is a flexible generalization of ordinary linear regression that allows for the response variable to have an error distribution other than the normal distribution.

General linear model (GLM) can be a general multivariate regression model. This GLM is a compact way of simultaneously writing several multiple linear regression models.

Life insurance is a contract between an insurance policy holder and an insurer or assurer, where the insurer promises to pay a designated beneficiary a sum of money (e.g. a benefit) upon the death of an insured person (e.g. a policy holder).

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, logistic regression, and/or sparse dictionary learning. Random forests (RF) (e.g. random decision forests) are an ensemble learning method for classification, regression, and other tasks, that operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Omnichannel is a cross-channel content strategy that organizations use to improve their user experience and drive better relationships with their audience across points of contact.

Principal component analysis (PCA) is the process of computing the principal components and using them to perform a change of basis on the data, sometimes using only the first few principal components and ignoring the rest. Principal components of a collection of points in a real coordinate space are a sequence of p unit vectors, where the ith vector is the direction of a line that best fits the data while being orthogonal to the first i-1 vectors. Here, a best-fitting line is defined as one that minimizes the average squared distance from the points to the line. These directions constitute an orthonormal basis in which different individual dimensions of the data are linearly uncorrelated.

Random forests or random decision forests are an ensemble learning method for classification, regression, and other tasks that operates by constructing a multitude of decision trees at training time. For classification tasks, the output of the random forest is the class selected by most trees. For regression tasks, the mean or average prediction of the individual trees is returned.

Example Methods

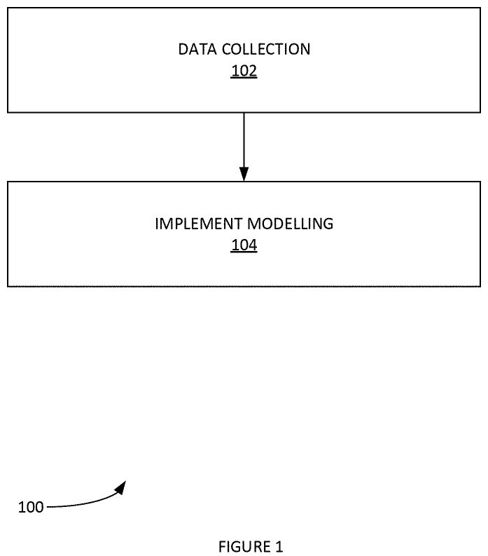

FIG. 1 illustrates an example process 100 for data set generation and modelling for determining a LifeScore, according to some embodiments. In step 102, process 100 can implement data collection. Process 100 can combine two different kinds of data sets in its models (e.g. see step 104).

In a first data set, process 100 can obtain a collection of all possible socioeconomic, demographic, health behavior and healthcare related, disease propensities, environmental attributes, social cohesion, civic engagement, and other metrics and data points associated with localities and neighborhoods.

This is to generate locality-age-gender specific life expectancy, mortality, and other health outcome related LifeScores and health scores. As used herein, it is noted that a LifeScore can be the result of a life-outcome model, and that the life-outcome model is, in turn, an outcome of inter alia: socioeconomic features, lifestyle/behavioral factors, environmental factors, etc. It is noted that an objective of this metric is for purposes of information. LifeScores can be determined at the county level. Accordingly, in some example embodiments, LifeScores can be generated across 3000+ counties in the United States for different age groups and genders respectively.

The underlying motivation and rationale here is that there are external, social determinants of life and health. The sources include, inter alia: Centers for Disease Control, National Center for Health Statistics, the National Institutes of Health, the American Medical Association, American Hospital Association, Bureau of Labor Statistics, to socio-economic and demographic sources, such as the US Census, the American Community Survey, the American Time Use Survey, County Business Pattern, etc. Process 100 can incorporate life expectancy data by age and by county into these scores.

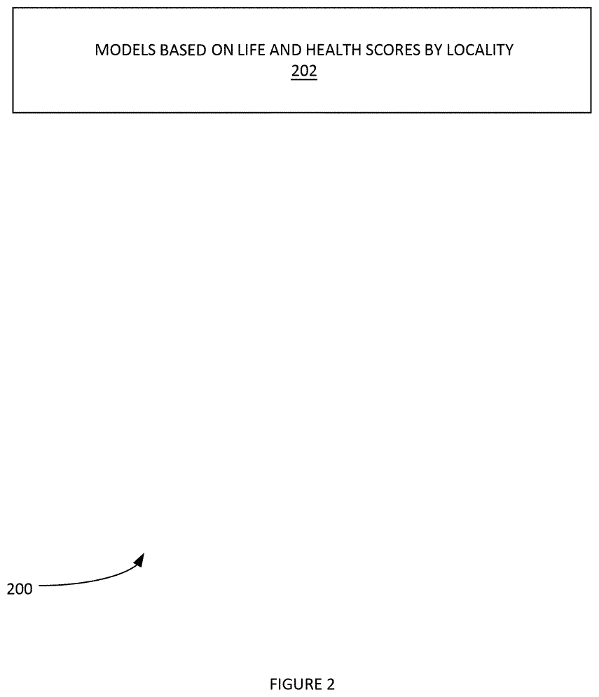

In step 104, process 100 can implement modelling. FIG. 2 illustrates an example process 200 for model implementation according to some embodiments. It is noted that machine learning process provide infra can be utilized by step 104 and process 200.

In step 202, process 200 can first determine models on life and health scores by locality. Process 200 can generate indices and/or principal components and/or combinations thereof that reflect life expectancy, health outcomes, disease propensity, health behavior, and/or other correlated metrics at the locality level. The models can incorporate a wide range of variables listed over and above the purely medical and healthcare ones. The following equation can be utilized by process 200:

LIFESCORE=WEIGHTED COMBINATION/INDEX (LIFE EXPECTANCY MODEL, POOR HEALTH MODEL, COMMON CAUSE OF DEATH MODELS BOTH NATURAL AND ACCIDENT) EACH INDIVIDUAL MODEL=FUNCTION (HEALTHCARE, ENVIRONMENT, SOCIO-ECONOMIC, OTHER EXTERNAL VARIABLES).

Example Machine Learning Implementations

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning. Random forests (RF) (e.g. random decision forests) are an ensemble learning method for classification, regression, and other tasks, that operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, that is a set of examples used to fit the parameters (e.g. weights of connections between neurons in artificial neural networks) of the model. The model (e.g. a neural net or a naive Bayes classifier) is trained on the training dataset using a supervised learning method (e.g. gradient descent or stochastic gradient descent). In practice, the training dataset often consist of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in a neural network). Validation datasets can be used for regularization by early stopping: stop training when the error on the validation dataset increases, as this is a sign of overfitting to the training dataset. Finally, the test dataset is a dataset used to provide an unbiased evaluation of a final model fit on the training dataset. If the data in the test dataset has never been used in training (e.g. in cross-validation), the test dataset is also called a holdout dataset.

Additional Example Computer Architecture and Systems

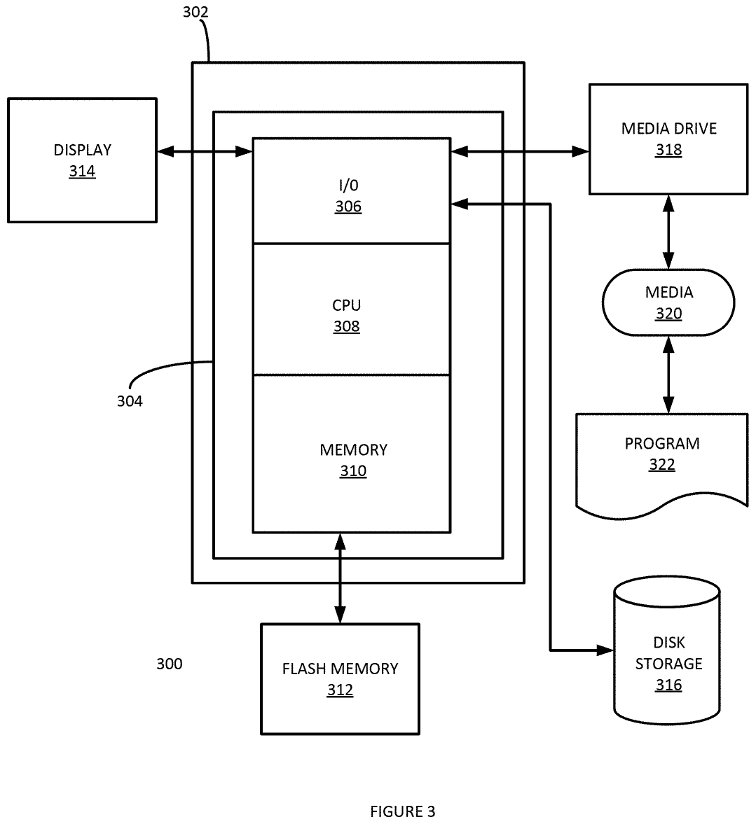

FIG. 3 depicts an exemplary computing system 300 that can be configured to perform any one of the processes provided herein. In this context, computing system 300 may include, for example, a processor, memory, storage, and I/O devices (e.g., monitor, keyboard, disk drive, Internet connection, etc.). However, computing system 300 may include circuitry or other specialized hardware for carrying out some or all aspects of the processes. In some operational settings, computing system 300 may be configured as a system that includes one or more units, each of which is configured to carry out some aspects of the processes either in software, hardware, or some combination thereof.

FIG. 3 depicts computing system 300 with a number of components that may be used to perform any of the processes described herein. The main system 302 includes a motherboard 304 having an I/O section 306, one or more central processing units (CPU) 308, and a memory section 310, which may have a flash memory card 312 related to it. The I/O section 306 can be connected to a display 314, a keyboard and/or other user input (not shown), a disk storage unit 316, and a media drive unit 318. The media drive unit 318 can read/write a computer-readable medium 320, which can contain programs 322 and/or data. Computing system 300 can include a web browser. Moreover, it is noted that computing system 300 can be configured to include additional systems in order to fulfill various functionalities. Computing system 300 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

Additional Processes

Generation of Datasets For Machine Learning Models Used to Determine a Lifescore

In some embodiments, LifeScores reflect and represent for each county, age group and gender, the prospects for quality of life and health relative to all counties in the United States. LifeScores can reflect life expectancy, mortality, and/or overall quality of health in the United States specific to location, age group and gender. A higher LifeScore implies higher life expectancy, lower mortality and better prospects for health and life for that age group and gender, in that county, relative to other counties. A LifeScore can range from 600 to 950. The higher the LifeScore the better the outlook for life and health in that county for that age group and gender. The LifeScores are based on a wide collection of all possible socioeconomic, demographic, lifestyle-behavioral and/or healthcare related features, disease propensities, environmental attributes, social cohesion, civic engagement and/or other metrics and data points associated with counties, and which have a bearing on health and life.

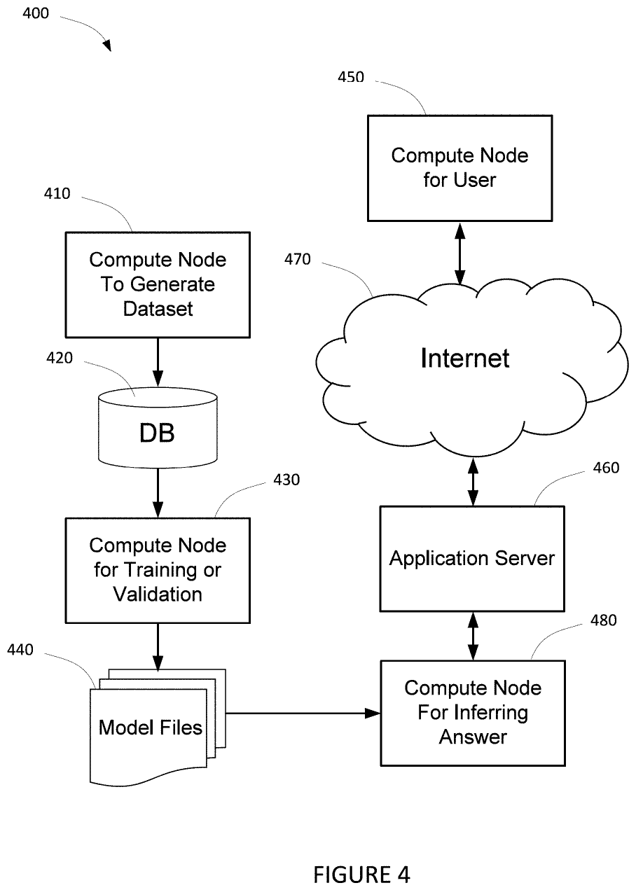

FIG. 4 is a schematic representation of an exemplary hardware environment 400 for generation of datasets for machine learning models used to determine a geo-location-based LifeScore, according to some embodiments. The hardware environment 400 includes a first compute node 410 that is employed to build a dataset for later use by machine-learning systems. In various embodiments the compute node 410 is a server but can be any computing device with sufficient computing capacity such as a server, personal computer, or smart phone. The compute node 410 stores the dataset to a database 420. More specifically, first compute node 410 builds a data set from various example sources, including, inter alia: United States Census Bureau server(s), American Community Survey ACS server(s), National Institutes of Health server(s), Center for Disease Control server(s), Bureau of Labor Statistics server(s), Dept of Transportation server(s), server(s) associated with data including Risk Surveys, National Centers for Environmental Information, National Center for Health Statistics—Mortality Files, U.S. Congress, Joint Economic Committee, Social Capital Project. “The Geography of Social Capital in America.”, server(s) associated with data including 2018. The 2020 County Health Rankings, server(s) associated with data including, server(s) associated with data including the Behavioral Risk Factor Surveillance System, County Business Patterns, FRED database Federal Reserve, Data.gov, Economic Research Service, U.S. department of agriculture server(s), Federal Interagency Forum on Aging-Related Statistics server(s), Public Use Microdata Samples (PUMS) server(s), Environmental Protection Agency server(s), etc. This data is stored in database 420.

First compute node 410 can use acquired data to provide values for specified feature variables. Features variables can be at the level of a county (e.g. in median, mean or percentage share form), and/or other geographic area that is a sufficiently large geography to ensure privacy issues are not a problem. Example feature variables include, inter alia: Population Med_income, Unemployment_pct_Unemployed, Mortality_rate, Adult_Smoking_pct_Smokers, Adult_Obesity_pct_Obese, Physical_Inactivity_pct_Physically_Inactive, Access_to_Exercise_Opportunities_pct_With_Access, Excessive_Drinking_pct_Excessive_Drinking, Alcohol_Impaired_Driving_Deaths_pct_Alcohol_Impaired, Primary_Care_Physicians_PCP_Rate, Food_Environment_Index, Violent_Crime_Violent_Crime_Rate, Injury_Deaths_Injury_Death_Rate, Violent_Crimes_p_100_000, Homicide_Rate, Drug_Overdose_Mortality_Rate, Motor_Vehicle_Mortality_Rate, Firearm_Fatalities_Rate, Informal civic engagement, etc.

It is noted that all data is at the level of a county: for example, average or median income, share or percent of people with college education etc. It is also noted that while most of the variables are self-explanatory and can be used as a percentage share of the population. An example list/dictionary of example feature variable definitions are now provided:

Access to exercise opportunities: Percentage of population with adequate access to locations for physical activity; includes parks, recreational areas;

Food environment index: Index of factors contributing to a healthy food environment based on access and proximity to grocery stores, expenditures on fast foods, food prices etc.;

Informal civic engagement: An index reflecting participation in both formal and informal activities, such as volunteering, participation in group activities, membership in clubs, recreational group activities etc.;

Frequent physical or mental distress: Percentage of adults reporting 14 or more days of poor physical or mental health by month;

Physical inactivity: Percentage of adults aged 20 and over reporting no physical activity.

There are two key sources of index data (e.g. non-primary data, survey data, and data combining many features into one), such as Food environment index, Access to exercise opportunities and other such transformed composite data features (e.g. see https://www.countyhealthrankings.org/).

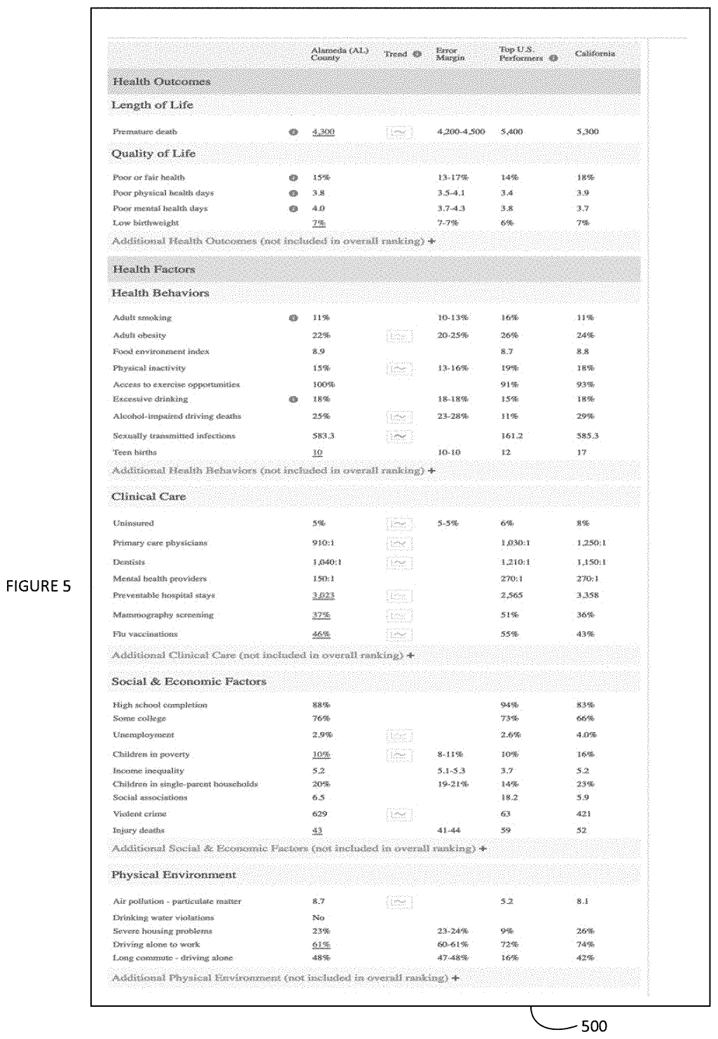

FIG. 5 illustrates an example of a webpage snapshot 500 of a an example of the county-wise data frames that can be utilized herein, according to some embodiments.

Another source for non-primary data can be the Social Capital Project of the Joint Economic Committee of the US Senate. The project is a multi-year research effort that investigates the “evolving nature, quality, and importance of our associational life”. “Associational life” is understood as the panoply of social networks, relationships, and linkages we enjoy in our communities, neighborhoods, localities, and cities, and which come into play in the pursuit of common, public, social tasks, objectives, and endeavors.

A second compute node 430, which can be the same compute node as first compute node 410, in some embodiments, accesses the database 420 in order to utilize the dataset to train deep learning models to produced trained model files 440. The second compute node 430 can optionally also validate deep learning models.

A user employing a third compute node 450 can upload an image or video, including a target therein, to an application server 460 across a network like the Internet 470, where the application server 460 hosts a machine-learning engine and a LifeScore engine. Machine-learning engine and a LifeScore engine manage the generation of machine learning models used and a geo-location-based LifeScore upon a request from a user.

In response to a request from the compute node 450, such as a mobile phone or PC, to calculate a LifeScore, the application server 460 connects the third compute node 450 to a fourth compute node 480, which can be the same compute node as either the first or second compute nodes 410, 430, in some embodiment. Compute node 480 uses the model files 440 to infer answers to the queries posed by the compute node 450 and transmits the answers back through the application server 460 to the compute node 450. Application server 460

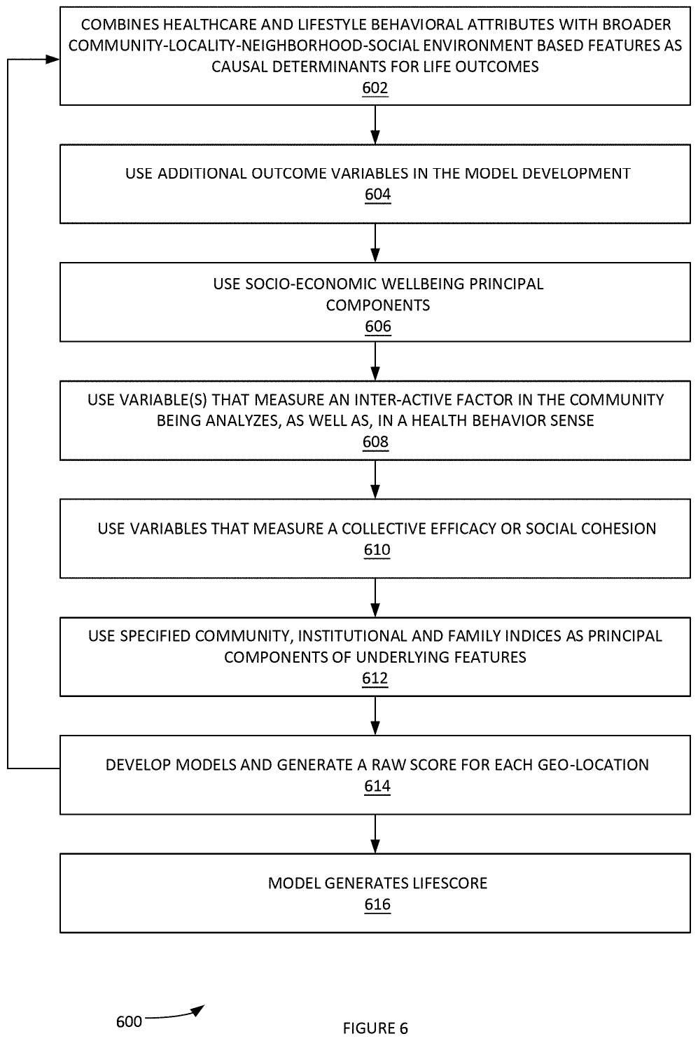

FIG. 6 illustrates an example process 600 for generation of datasets for machine learning models used to determine a geo-location-based LifeScore, according to some embodiments. Process 600 can use a large number of various algorithms and statistical methods: GLM, GBM, Random Forest Regressions, PCA, etc. These can be built on a range of features. It is noted that LifeScores are a form of index creation and involve a number of steps provided for herein. LifeScores can be an integrated, holistic, broad-based approach related to the idea that in addition to various factors there are social, external, environmental determinants of health and life expectancy.

In step 602, process 600 uses a modeling-methodological approach (e.g. see process 400, etc.) that combines healthcare and lifestyle behavioral attributes with broader community-locality-neighborhood-social environment-based features as causal determinants for life outcomes.

In addition to standard healthcare metrics assessed in step 602 (e.g. mortality and life expectancy at each age cohort and by gender and location, etc.), in step 604 process 600 also uses additional outcome/target variables in the model development. These can include, inter alia:

Self assessed poor health: age adjusted data on percentage of adults reporting fair or poor health;

Poor physical health days, Poor mental health days: these can be the average number of physically or mentally unhealthy days in the past 30 days;

Frequent physical distress: this can be the percentage of adults reporting 14 or more days of poor physical health in the last month;

Self assessed poor health Poor physical health days Frequent physical distress In step 606.

Process 600 uses socio-economic wellbeing principal components (e.g. as indices) developed on data to on, inter alia:

Income, employment, education, the physical environment, that includes air and water quality as well as housing and transit related variables; and

Violent crime rate, injury deaths and firearm fatalities.

In step 608, process 700 also uses variable(s) that are measures/indices/multi-factor combinations reflecting different characteristics of the community being analyzed in a holistic health and well-being sense. Examples of these variables, include, inter alia:

Access to healthy foods and exercise opportunities;

Civil Society and Social Capital Principal Components developed on data/indices on , inter alia: Social support or social capital metrics including social associations rate, which are say number of non-profit membership associations per 10,000 population a measure of civil society;

Share of Children and single parent households;

Community connectedness and safety; and

Family and social support metrics.

In step 610, process 600 can use variables that measure collective efficacy or social cohesion. These can reflect what residents are willing to do to improve their neighborhoods; joint capabilities to organize and execute action required to produce better outcomes for the community.

In step 612, process 600 can use specified community, institutional and family indices as principal components of underlying features. These underlying features can include, inter alia: births to unmarried women, percent of children with single parents, voting rate, mail-in census rate, survey of confidence in institutions etc.

In step 614, process 600 develops and uses predictive models that use various algorithms and statistical methods: GLM, GBM, Logistic Regressions using PCAs and individual features on a range of life and health outcomes, etc. These can then be combined using both fitted values and residual analysis to generate an overarching raw LifeScore. Process 600 can generate weights based on a goodness of the fit of various models.

In step 616, the raw score is then mapped onto a scaled, calibrated LifeScore depending on the distribution characteristics of the Raw Score and to fit into a 600-950 scale. Process 600 can be repeated for each geo-location (e.g. county, etc.).

Process 600 can utilize the following equations to generate its output:

EACH INDIVIDUAL MODEL WITH OUTPUT SUCH AS LIFE EXPECTANCY (MORTALITY) FOR THAT GENDER, AGE GROUP AND COUNTY=FUNCTION (HEALTHCARE, ENVIRONMENTAL, SOCIO-ECONOMIC, LIFESTYLE-BEHAVIORAL, DEMOGRAPHIC OTHER EXTERNAL VARIABLES): and

LIFESCORE=WEIGHTED COMBINATION/INDEX (LIFE EXPECTANCY MODEL, POOR HEALTH MODEL, MORTALITY AND COMMON CAUSE OF DEATH MODELS BOTH NATURAL AND ACCIDENT).

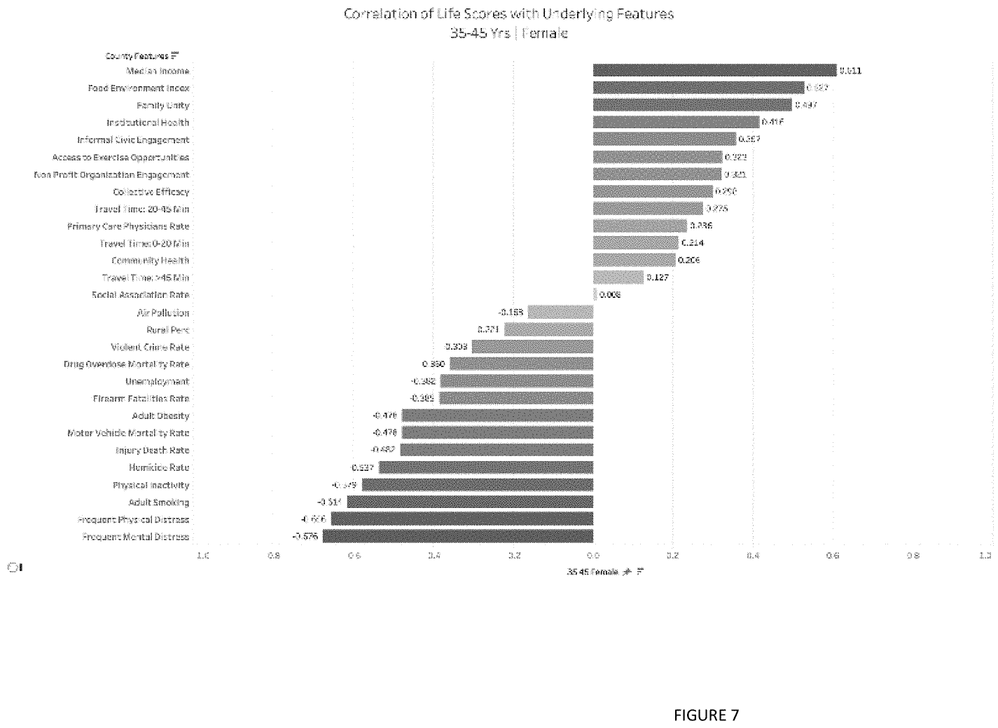

FIG. 7 illustrates an example screen shot 700 of correlations for a LifeScore, according to some embodiments. More specifically, screen shot 700 shows the correlations of LifeScore for a 35- to 45-year-old female averaged out over the entire United States. Screen shot 700 also illustrates various underlying features that can be input into the calculation of the LifeScore. As a measure of validation, the median income, the food environment index, the measure of Access to exercise opportunities etc. are positively correlated with the LifeScore. At the same time the violent crime rate, the unemployment rate, adult obesity rate, adult smoking rate, the frequent physical distress metric all have a negative impact on LifeScore.

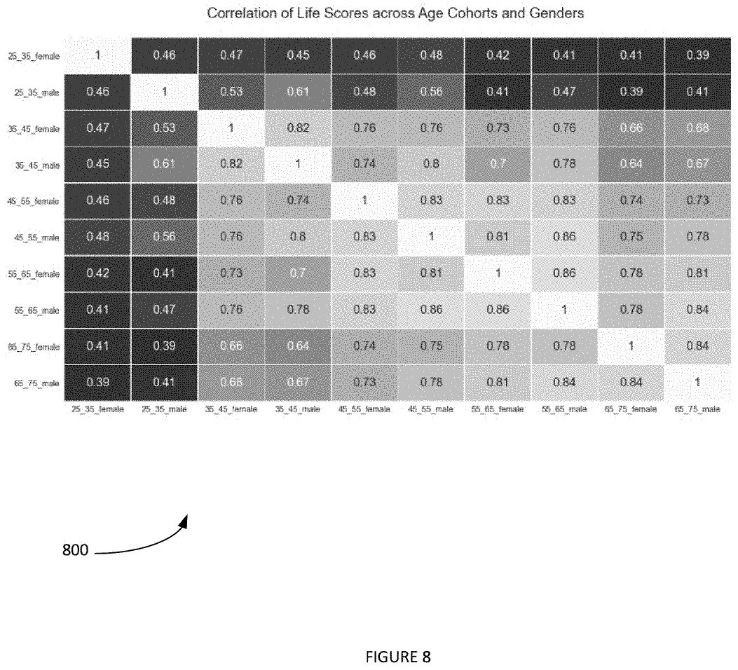

FIG. 8 illustrates an example chart illustrating how LifeScores are correlated across age groups and gender, according to some embodiments.

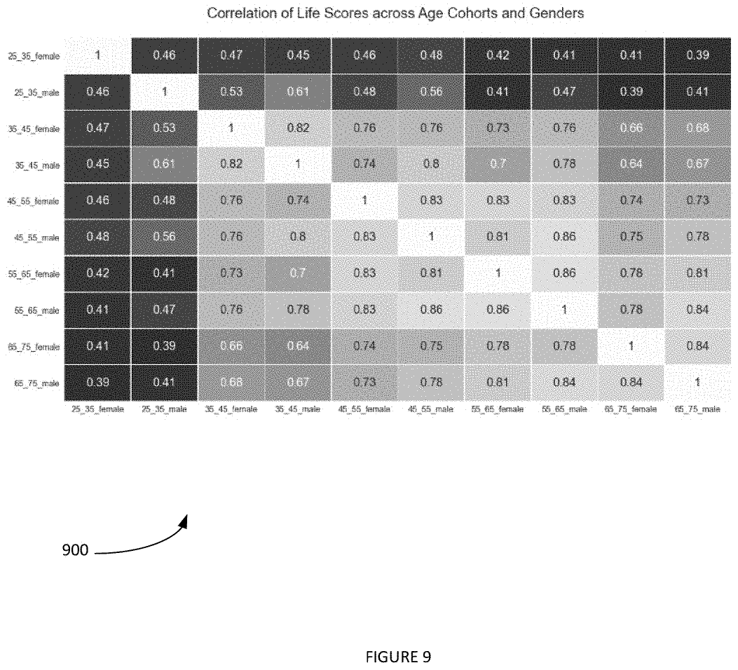

FIG. 9 illustrates screenshot of LifeScore web map, according to some embodiments.

Conclusion

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

1. A computerized method for generation of datasets for machine learning models used to determine a geo-location-based LifeScore comprising:
implementing a machine learning modeling process to combine county specific healthcare attribute variables, lifestyle behavioral attribute variables, demographic, socio-economic, socio-cultural-networking variables to generate a life outcome model of a county;
updating the life outcome model based on a self assessed poor health variable value, a poor physical health days variable value, a frequent physical distress variable value;
using a set of socio-economic wellbeing principal components to update the life outcome model;
generating a community well-being index of the locality to update the life outcome model;
using a set of variables that measure collective efficacy or social cohesion to update the life outcome model;
using a specified community, institutional and family index to update the life outcome mode; and
using the life outcome model to generate a LifeScore.

2. The computerized method of claim 1, wherein lifestyle behavioral attribute variable comprises a mortality and a life expectancy at each age cohort and by a gender and a location.

3. The computerized method of claim 1, wherein the set of using the set of socio-economic wellbeing principal component comprises an average income, share of college education, and other “standard of living” related variables, which are correlated at the level of a geo-location.

4. The computerized method of claim 3, wherein the set of using the set of socio-economic wellbeing principal component comprises a physical environment principal component comprising an air and water quality index.

5. The computerized method of claim 4, wherein the physical environment principal component comprises a violent crime rate, a death rate, and a firearm fatality rate.

6. The computerized method of claim 1, wherein the community well-being index to update the life outcome model reflecting different characteristics of the community being analyzed in a holistic health and well-being sense.

7. The computerized method of claim 6, wherein the community well-being index comprises an access to healthy foods and exercise opportunities variable value.

8. The computerized method of claim 7, wherein the community well-being index comprises a civil society and social capital variable value that is developed on a social support and a social capital metric.

9. The computerized method of claim 8, wherein the social support and a social capital metric comprises a social associations rate, a share of children and single parent households rate; a community connectedness and safety rate; and family and social support rate.

10. The computerized method of claim 9, wherein the set of variables that measure collective efficacy or social cohesion reflect what residents are willing to do to improve their neighborhoods.

11. The computerized method of claim 1, where in the specified community, institutional and family index is generated from an average number of births to unmarried women in the locality, percent of children with single parents in the locality variable value, a voting rate in the locality variable value, a mail-in census rate in the locality variable value, and a survey of confidence in institutions in the locality variable value.

12. The computerized method of claim 1, wherein the life outcome model is generate using one or more machine-learning predictive models using GLM, GBM, or a Logistic Regression using both principal component analysis and individual features.

13. The computerized method of claim 12, wherein the one or more machine-learning predictive models are combined using both fitted values and residual analysis to generate the LifeScore.

14. The computerized method of claim 13, wherein the LifeScore is mapped onto a scaled, calibrated LifeScore and to fit into a 600-950 scale.

15. The computerized method of claim 1 wherein the locality comprises a county.
